(19) United States
(12) Patent Application Publication
(10) Pub. No.: US 2009/0011946 A1
(43) Pub. Date: Jan. 8, 2009

(54) Use of Sequence Specific Polymers in Chemical Detection

(75) Inventors: Arun Majumdar, Orinda, CA (US); Mark O. Trulson, San Jose, CA (US)

Correspondence Address: BUCHANAN, INGERSOLL & ROONEY PC, POST OFFICE BOX 1404, ALEXANDRIA, VA, 22313-1404, US

(73) Assignee: Kalinex, Berkeley, CA (US)

(21) Appl. No.: 11/793,576

(22) PCT Filed: Dec. 20, 2005
(86) PCT No.: PCT/US2005/046663

§ 371 Date: May 30, 2008

Related U.S. Application Data
(60) Provisional application No. 60/638,327, filed on Dec. 20, 2004.
(60) Provisional application No. 60/670,473, filed on Apr. 11, 2005.

(51) Int. Cl. C40B 30/04; B01J 19/00; G01N 21/64; G01N 27/00; C40B 40/14; C08G 73/00; G01N 33/566

(52) U.S. Cl. 506/9; 422/68.1; 422/82.01; 422/82.08; 528/422; 436/501; 506/20

(57) ABSTRACT

A method for chemical detection is provided. In one aspect, the method comprises exposing a sample to a sequence specific polymer under conditions such that an analyte in the sample binds to the polymer. Binding of the analyte to the sequence specific polymer results in a change in a property of the sequence specific polymer that is transduced to a response transduction medium, which generates a detectable response. Another aspect provides a detection device comprising the sequence specific polymer and response transduction medium.

### CROSS REFERENCE TO RELATED APPLICATIONS

This application claims priority to U.S. Provisional Application Ser. No. 60/638,327 filed Dec. 20, 2004, and U.S. Provisional Application Ser. No. 60/670,473, filed Apr. 11, 2005, the disclosures of which are incorporated herein by reference.

### FIELD OF THE INVENTION

This invention generally relates to the field of chemical detection and, more particularly, relates to methods and devices for use of sequence specific polymers for chemical detection.

### BACKGROUND

The ability of man-made devices to selectively detect the presence of specific structural elements associated with an analyte depends on the phenomenon of specific chemical recognition. In general, the target or “analyte” molecule either binds or induces a change to a binding element such as a receptor molecule. Achieving high selectivity for the target in binding reactions reduces false positives and negatives in any detection system. However, there is often a limited availability, or no availability, of appropriate binding elements for a desired target analyte. Therefore, an unaddressed need exists in the industry to address the aforementioned deficiencies and inadequacies.

### SUMMARY OF THE INVENTION

The present invention relates to methods and devices for use of sequence specific polymers for chemical detection. In one embodiment, the invention provides a method of detecting an analyte in a sample. The method comprises exposing a sample to a sequence specific polymer under conditions such that an analyte in the sample binds to the sequence specific polymer. Binding of the analyte to the sequence specific polymer results in a change in a property of the sequence specific polymer that is transduced to a response transduction medium which generates a detectable response. The response is then detected.

In another embodiment of the invention, a method of detecting an analyte in a sample is provided which comprises the following. A sample is exposed to a sequence specific polymer under conditions such that an analyte in the sample binds to the sequence specific polymer. Binding of the analyte to the sequence specific polymer results in a high local concentration of analyte which causes a change in a property of one or both of:

(a) an ingredient other than the sequence specific polymer in a sequence specific polymer-containing composition, or

(b) a component of a response transduction medium.

The change in property is transduced to the response transduction medium which generates a detectable response. The response is then detected.

In yet another embodiment, the invention provides a detection device. The device comprises a sequence specific polymer and a response transduction medium. The device has the characteristic that binding of an analyte to the sequence specific polymer causes a change in a property of the sequence specific polymer that is transduced to the response transduction medium and results in a detectable signal.

Another embodiment of the invention provides a detection device comprising a sequence specific polymer and a response transduction medium. The device has the characteristic that binding of an analyte to the sequence specific polymer results in a high local concentration of analyte which causes a change in a property of one or both of:

(a) an ingredient other than the sequence specific polymer in a sequence specific polymer-containing composition, or

(b) a component of the response transduction medium.

The change in property is transduced to the response transduction medium and results in a detectable signal.

Another embodiment of the invention provides a sequence specific polymer for use in analyte detection, wherein binding of an analyte to the sequence specific polymer causes a change in the sequence specific polymer which is capable of being transduced to a detectable response.

In another embodiment, the invention provides a method for identifying a sequence specific polymer. The method comprises:

(a) providing a preselected target analyte molecule;

(b) providing a plurality of monomers;

(c) identifying monomers that individually, or in combination, interact with the target analyte or portion of the target analyte;

(d) assembling the monomers identified in c) into at least one sequence specific polymer comprising a specific sequence and length of monomers, wherein the polymer comprises at least two monomers;

(e) contacting the sequence specific polymer with the preselected target analyte molecule; and

(f) identifying a sequence specific polymer that interacts with the preselected target analyte molecule.

Other systems, methods, features and advantages of the present invention will be or become apparent to one with skill in the art upon examination of the following drawings and detailed description. It is intended that all such additional systems, methods, features and advantages be included within this description, be within the scope of the present invention, and be protected by the accompanying claims.

### BRIEF DESCRIPTION OF DRAWINGS

FIG. 1 depicts a flow diagram for assembling and screening sequence specific polymers.

FIG. 2 depicts a flow diagram for assembling and screening sequence specific polymers.

FIG. 3 depicts a flow diagram for assembling a device that includes sequence specific polymers.

FIG. 4 depicts a flow diagram for assembling a device that includes sequence specific polymers that minimize the effects of interfering molecules contained in a sample comprising a target analyte.

FIG. 5 depicts devices, device chips, and device systems.

### DESCRIPTION OF PREFERRED EMBODIMENTS

Detection of chemical particulates, contaminants, or vapors has important applications in medicine, public safety and national security for detecting target molecules associated with, e.g., a particular disease, environmental pollutant, explosive or illegal drug. Accordingly, this invention relates to the field of chemical detection using devices that are sensitive to single molecular species or closely related families of species. Polymers that have increased affinity for a target analyte are provided, as well as detection devices comprising those polymers. Also provided are methods for detecting a target analyte by using the detection devices. In addition, methods for identifying such polymers are provided herein. The devices may be designed to detect target molecules in the gas phase, as well as condensed phases, in the presence of potentially interfering backgrounds heterogeneous populations of molecular species.

Further provided herein are methods and compositions used to generate and screen polymers comprising at least two different monomer species, with a pre-determined length and specific sequence of monomers for binding to an analyte molecule of interest (also referred to herein as “target analyte” or “preselected analyte”).

### Sequence Specific Polymers

The sequence specific polymers (SSP) of the present invention are any polymers with a predetermined, defined sequence, which are composed of at least two monomer units, and which may exhibit a variety of chemical functionalities. Although it is desired that a preparation of sequence specific polymers contain polymers of identical length and sequence, the preparation may contain impurities of polymers having minor differences in length and sequence as long as the sequence specific polymer preparation can be used to identify a target analyte. Typically, the sequence specific polymers have different monomer units. The sequence specific polymers include both biopolymers and non-biopolymers. Biopolymers include, for example, proteins, nucleic acids and polysaccharides. Proteins include naturally-occurring variants as well as proteins with inserted, deleted or mutated amino acid residues, truncated proteins, and fusion proteins. Engineered proteins which have no apparent resemblance to any naturally-occurring protein but which nevertheless have a specific binding affinity for a target analyte may also be used. Nucleic acids include DNA, RNA and DNA-RNA hybrids. The nucleic acids encode any of the aforementioned proteins and include nucleic acids with naturally-occurring sequences, as well as nucleic with degenerate sequences. Non-biopolymers are polymer types not found in nature. Examples include optionally substituted polyethylenes, polypropylenes, polystyrenes, polycarbonates, polyterephthalates, polysilanes, polyurethanes and polyethers. Peptoids (i.e., poly(N-substituted glycine)) are another example of non-biopolymers. Any and all embodiments of the invention are contemplated as being carried out with biopolymers, non-biopolymers, or any subclass within biopolymers or non-biopolymers. In one embodiment, a polymer may exhibit high sequence complexity by incorporating a wide variety of monomeric units, and can be synthesized in bulk to have identical sequences and lengths.

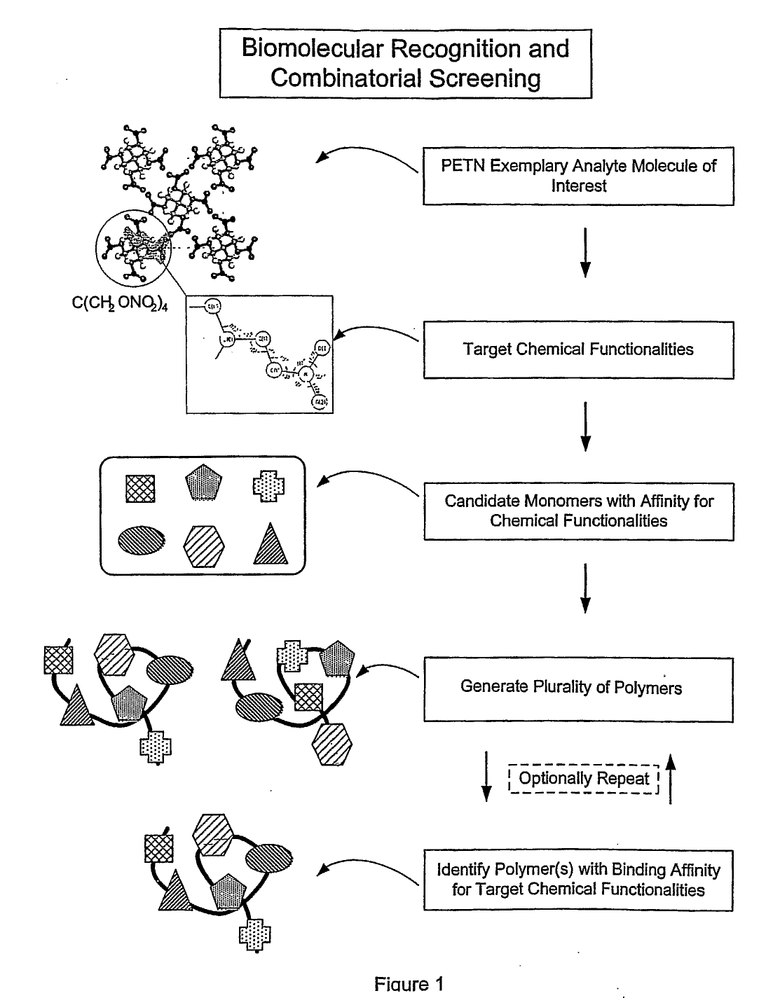

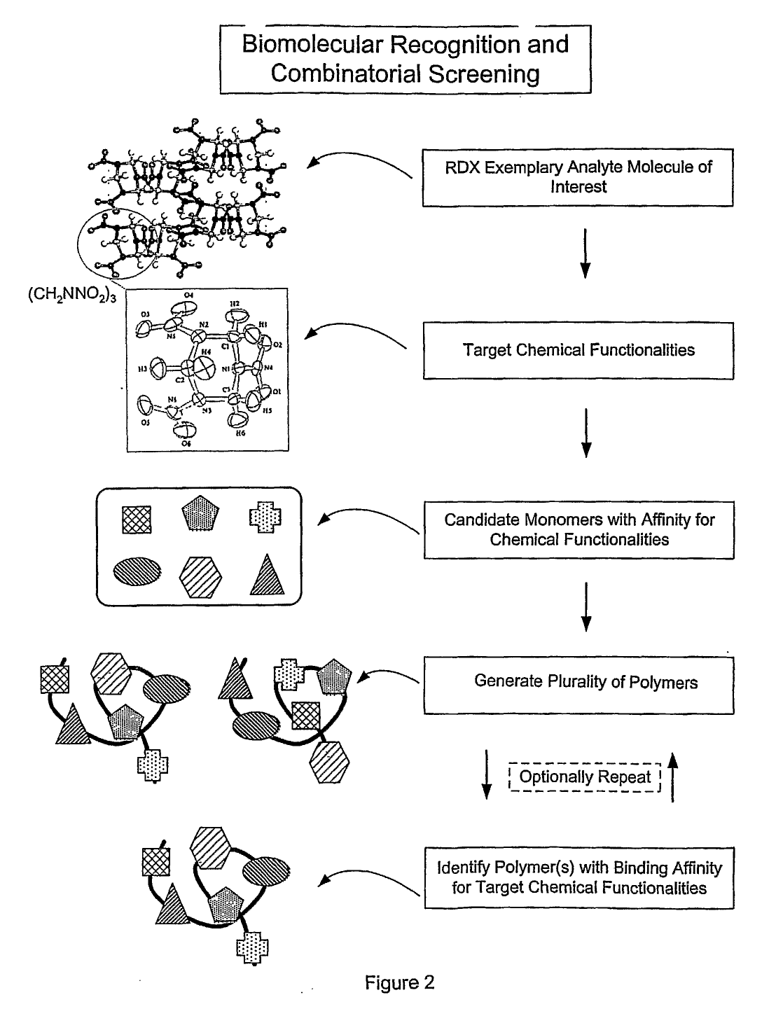

FIG. 1 and FIG. 2 provide a general description of methods and compositions used to generate and screen polymers comprising at least two different monomer species, with a pre-determined length and specific sequence of monomers (i.e., SSP) for binding to an analyte molecule of interest (also referred to herein as “target analyte” or “preselected analyte”). Generating monomer units for inclusion in a synthetic polymer may include choosing a particular monomer based upon its affinity for various chemical functionalities present in a preselected analyte molecule of interest. The exemplary molecule of interest included in FIG. 1 is PETN, a chemical component of an explosive material. The exemplary molecule of interest included in FIG. 2 is RDX which is also a chemical component of an explosive material. However, it is understood that sequence specific polymers may be designed that have an affinity for, or are predicted to have an affinity for, a particular chemical functionality associated with practically any type of analyte molecule. Such analyte molecules include proteins, nucleic acids, lipids, or any other organic or non-organic molecule. All that is required is the availability of structural information for chemical functionalities associated with the analyte molecule of interest.

Based upon this information, candidate monomers with an affinity for particular chemical functionalities can be chosen from a set of monomers and assembled into sequence specific polymers. The polymers can then be screened against the functionality to identify those polymers that have the highest affinity for the functionality. The sequence of monomers in a polymer of the invention may be selected by computational analysis. For example, the sequence of monomers may be selected based upon structural information available for a naturally-occurring recognition site for a target analyte molecule.

A “polymer,” as used herein, is a material formed by combining units, i.e., monomers, into chains. Examples of polymers are starch (which has many sugar units), polyethylene (which has many ethylene units) and polystyrene (which has many styrene units). Synthetic polymers may be formed by addition or condensation polymerization of monomers. If two or more different monomers are involved, a “copolymer” is obtained. With regard to this document, the term “polymer” is sometimes used interchangeably with the terms copolymer, multimer and multi-block polymer. Accordingly, it is understood that a “polymer with a pre-determined length and specific sequence of monomers” (i.e., SSP), as used herein to describe a molecule that binds to a target analyte molecule of interest, encompasses polymers, copolymers, multimers, and multi-block polymers.

In some embodiments, a polymer can include discrete “blocks” of monomers. A “block” is a region of a polymer composed primarily of (i) a single monomer type, (ii) a well-defined repeated motif, or (iii) a well-defined alternation of motifs (in which case the shorter alternating motifs can be linked together as a longer repetitive motif). Generally a block is comprised of no more than about 80 monomers (e.g., 3, 5, 10, 20, 30, 50, or 75 monomers). Each block can include either a single type of monomer (and therefore is also called a “homoblock”), or two or more (e.g., 3, 4, 5, or more) chemically different types of monomers arranged in a pattern to form substructures (also referred to as “motifs”) repeated throughout the block (and therefore is also called a “heteroblock”). Accordingly, a “multi-block” polymer includes more than one “block” or “region” of monomers as described above.

In other embodiments, polymers, copolymers, or multi-block polymers are linked together through linkers to form “multimers.” The term “linker” is used herein to indicate a moiety or group of moieties that joins or connects two or more discrete polymers. The linker moiety is typically a substantially linear moiety. Suitable linkers include polypeptides, polynucleic acids, peptide nucleic acids and the like. Suitable linkers also include optionally substituted alkylene moieties that have one or more oxygen atoms incorporated in the carbon backbone. Typically, the molecular weight of the linker is less than about 2000 daltons. More typically, the molecular weight of the linker is less than about 1500 daltons and usually is less than about 1000 daltons. The linker can be small enough to allow the discrete separate polymers to cooperate, e.g., where each of the discrete separate polymers in a multimer binds to the same target analyte molecule via separate binding sites. A multimer can include a plurality of polymers or copolymers, a plurality of multi-block polymers, or any combination thereof.

The term “monomer” is used herein to refer to a single molecule that has the ability to combine with identical or other molecules in a process known as polymerization. Monomers described herein are chosen based upon their ability to interact with specific chemical groups present on a target analyte molecule. The polymerization reaction may be a dehydration or condensation reaction (due to the formation of water (H₂O) as one of the products) where a hydrogen atom and a hydroxyl (—OH) group are lost to form H₂O and an oxygen molecule bonds between each monomer unit. Note that polymers built from monomers can also be called dimers, trimers, tetramers, pentamers, hexamers, octamers, 10-mers, 15-mers, 20-mers, etc. if they have 2, 3, 4, 5, 6, 8, 10, 15 or 20 monomer units, respectively.

It is understood that the term “monomer” includes any chemical group that can be assembled into a polymer. A wide variety of monomers may be used for synthesizing a polymer in accordance with the principles of the present invention. For example, a polymer of the invention may be composed of monomers that have, for example, affinity property groups, hydrophilic groups, and/or hydrophobic groups pendant from their backbones. Accordingly, a polymer may include side chains “R” pendant from a structurally repetitive backbone. Exemplary backbones with side chains include:

structure=—(CO—N(—R)—CH₂)—;

structure=—(O—Si(—CH₃)(—R))—;

structure=—(CH₂—CH(—R)—CO—NH)—;

structure=—(CH₂—CH(—R)—O)—; and

structure=—(CH₂—C₆H₄—CO—N(—R))—.

Exemplary backbones with side chains selected for hydrophobicity include:

structure=—(CH₂—CHR)—, or —(CH₂—CH₂—CHR)—; and

structure=—(CF₂—CFR), or —(CF₂—CF₂—CFR)—.

Exemplary backbones with side chains selected for hydrophilicity include:

structure=—(CH₂—CH(—CO—NHR))—.

When referring to polymers, the terms “hydrophilic” and “hydrophobic” are generally defined in terms of an HLB value, i.e., a hydrophilic lipophilic balance. A high HLB value indicates a hydrophilic compound, while a low HLB value characterizes a hydrophobic compound. HLB values are well known in the art, and generally range from 1 to 18.

Additional examples of suitable monomers include, but are not limited to, those described in the references cited in this written description and incorporated by reference herein. With regard to nomenclature pertinent to molecular structures, the following definitions apply:

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of one to six carbon atoms, preferably one to four carbon atoms. “Substituted alkyl” refers to alkyl substituted with one or more substituent groups. “Alkylene,” “lower alkylene” and “substituted alkylene” refer to divalent alkyl, lower alkyl, and substituted alkyl groups, respectively. The term “aryl” as used herein, and unless otherwise specified, refers to an aromatic substituent containing a single aromatic ring (monocyclic) or multiple aromatic rings that are fused together, linked covalently, or linked to a common group such as a methylene or ethylene moiety. The common linking group may also be a carbonyl as in benzophenone, an oxygen atom as in diphenylether, or a nitrogen atom as in diphenylamine. Preferred aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl in which at least one carbon atom is replaced with a heteroatom. The terms “arylene” and “substituted arylene” refer to divalent aryl and substituted aryl groups as just defined.

The term “heteroatom-containing” as in a “heteroatom-containing hydrocarbyl group” refers to a molecule or molecular fragment in which one or more carbon atoms is replaced with an atom other than carbon, e.g., nitrogen, oxygen, sulfur, phosphorus or silicon.

“Hydrocarbyl” refers to univalent hydrocarbyl radicals containing 1 to about 30 carbon atoms, preferably 1 to about 24 carbon atoms, most preferably 1 to about 12 carbon atoms, including branched or unbranched, saturated or unsaturated species, such as alkyl groups, alkenyl groups, aryl groups, and the like. The term “lower hydrocarbyl” intends a hydrocarbyl group of one to six carbon atoms, preferably one to four carbon atoms. The term “hydrocarbylene” intends a divalent hydrocarbyl moiety containing 1 to about 30 carbon atoms, preferably 1 to about 24 carbon atoms, most preferably 1 to about 12 carbon atoms, including branched or unbranched, saturated or unsaturated species, or the like. The term “lower hydrocarbylene” intends a hydrocarbylene group of one to six carbon atoms, preferably one to four carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substituent groups, and the terms “heteroatom-containing hydrocarbylene” and “heterohydrocarbylene” refer to hydrocarbylene in which at least one carbon atom is replaced with a heteroatom. If not otherwise indicated, “hydrocarbyl” indicates both unsubstituted and substituted hydrocarbyls, “heteroatom-containing hydrocarbyl” indicates both unsubstituted and substituted heteroatom-containing hydrocarbyls and so forth.

By “substituted” as in “substituted hydrocarbyl,” “substituted alkyl,” and the like, as alluded to in some of the aforementioned definitions, is meant that in the hydrocarbyl, alkyl, or other moiety, at least one hydrogen atom bound to a carbon atom is replaced with one or more substituents that are functional groups such as alkoxy, hydroxy, halo, nitro, and the like. Unless otherwise indicated, it is to be understood that specified molecular segments can be substituted with one or more substituents that do not compromise a compound's utility. For example, “succinimidyl” is intended to include unsubstituted succinimidyl as well as sulfosuccinimidyl and other succinimidyl groups substituted on a ring carbon atom, e.g., with alkoxy substituents, polyether substituents, or the like.

Any concentration ranges, percentage ranges, or ratio ranges recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. As used herein, the term “about” refers to +/−15% of any indicated structure, value, or range.

Additional non-limiting examples of monomers that can be used for preparing a polymer of the present invention include methylmethacrylate, other alkyl methacrylates, alkylacrylates, allyl or aryl acrylates and methacrylates, cyanoacrylate, styrene, alpha-methyl styrene, vinyl esters, including vinyl acetate, vinyl chloride, methyl vinyl ketone, vinylidene chloride, acrylamide, methacrylamide, acrylonitrile, methacrylonitrile, 2-acetamido acrylic acid; 2-(acetoxyacetoxy)ethyl methacrylate; 1-acetoxy-1,3-butadiene; 2-acetoxy-3-butenenitrile; 4-acetoxystyrene; acrolein; acrolein diethyl acetal; acrolein dimethyl acetal; acrylamide; 2-acrylamidoglycolic acid; 2-acrylamido-2-methyl propane sulfonic acid; acrylic acid; acrylic anhydride; acrylonitrile; acryloyl chloride; (R)-alpha-acryloxy-.beta., beta′-dimethyl-g-butyrolactone; N-acryloxy succinimide N-acryloxytris(hydroxymethyl) aminomethane; N-acryloly chloride; N-acryloyl pyrrolidinone; N-acryloyl-tris(hydroxymethyl)amino methane; 2-amino ethyl methacrylate; N-(3-aminopropyl)methacrylamide; (o, m, or p)-amino-styrene; t-amyl methacrylate; 2-(1-aziridinyl)ethyl methacrylate; 2,2′-azobis-(2-amidinopropane); 2,2′-azobisisobutyronitrile; 4,4′-azobis-(4-cyanovaleric acid); 1,1′-azobis-(cyclohexanecarbonitrile); 2,2′-azobis-(2,4-dimethylvaleronitrile); 4-benzyloxy-3-methoxystyrene; 2-bromoacrylic acid; 4-bromo-1-butene; 3-bromo-3,3-difluoropropane; 6-bromo-1-hexene; 3-bromo-2-methacrylonitrile; 2-(bromomethyl)acrylic acid; 8-bromo-1-octene; 5-bromo-1-pentene; cis-1-bromo-1-propene; beta-bromostyrene; p-bromostyrene; bromotrifluoro ethylene; (±)-3-buten-2-ol; 1,3-butadiene; 1,3-butadiene-1,4-dicarboxylic acid 3-butenal diethyl acetal; 1-butene; 3-buten-2-ol; 3-butenyl chloroformate; 2-butylacrolein; N-t-butylacrylamide; butyl acrylate; butyl methacrylate; (o,m,p)-bromostyrene; t-butyl acrylate; (R)-carvone; (S)-carvone; (−)-carvyl acetate; c is 3-chloroacrylic acid; 2-chloroacrylonitrile; 2-chloroethyl vinyl ether; 2-chloromethyl-3-trimethylsilyl-1-propene; 3-chloro-1-butene; 3-chloro-2-chloromethyl-1-propene; 3-chloro-2-methyl propene; 2,2-bis(4-chlorophenyl)-1,1-dichloro ethylene; 3-chloro-1-phenyl-1-propene; m-chlorostyrene; o-chlorostyrene; p-chlorostyrene; 1-cyanovinyl acetate; 1-cyclopropyl-1-(trimethylsiloxy)ethylene; 2,3-dichloro-1-propene; 2,6-dichlorostyrene; 1,3-dichloropropene; 2,4-diethyl-2,6-heptadienal; 1,9-decadiene; 1-decene; 1,2-dibromoethylene; 1,1-dichloro-2,2-difluoroethylene; 1,1-dichloropropene; 2,6-difluorostyrene; dihydrocarveol; (±)-dihydrocarvone; (−)-dihydrocarvyl acetate; 3,3-dimethylacrylaldehyde; N,N′-dimethylacrylamide; 3,3-dimethylacrylic acid; 3,3-dimethylacryloyl chloride; 2,3-dimethyl-1-butene; 3,3-dimethyl-1-butene; 2-dimethyl aminoethyl methacrylate; 2,4-dimethyl-2,6 heptadien-1-ol; 2,4-dimethyl-2,6-heptadienal; 2,5-dimethyl-1,5-hexadiene; 2,4-dimethyl-1,3-pentadiene; 2,2-dimethyl-4-pentenal; 2,4-dimethylstyrene; 2,5-dimethylstyrene; 3,4-dimethylstryene; divinyl benzene; 1,3-divinyltetramethyl disiloxane; 8,13-divinyl-3,7,12,17-tetramethyl-21H,23H-porphine; 8,13-divinyl-3,7,12,17-tetramethyl-21H,23H-propionic acid; 8,13-divinyl-3,7,12,17-tetramethyl-21H,23H-propionic acid disodium salt; 3,9-divinyl-2,4,8,10-tetraoraspiro[5,5]undecane; divinyl tin dichloride; 1-dodecene; 3,4-epoxy-1-butene; 2-ethyl acrolein; ethyl acrylate; 2-ethyl-1-butene; (±)-2-ethylhexyl acrylate; (±)-2-ethylhexyl methacrylate; 2-ethyl-2-(hydroxymethyl)-1,3-propanediol triacrylate; 2-ethyl-2-(hydroxymethyl)-1,3-propanediol trimethacrylate; ethyl methacrylate; ethyl vinyl ether; ethyl vinyl ketone; ethyl vinyl sulfone; (1-ethylvinyl)tributyl tin; m-fluorostyrene; o-fluorostyrene; p-fluorostyrene; glycol methacrylate (hydroxyethyl methacrylate); GA GMA; 1,6-heptadiene; 1,6-heptadienoic acid; 1,6-heptadien-4-ol; 1-heptene; 1-hexen-3-ol; 1-hexene; hexafluoropropene; 1,6-hexanediol diacrylate; 1-hexadecene; 1,5-hexadien-3,4-diol; 1,4-hexadiene; 1,5-hexadien-3-ol; 1,3,5-hexatriene; 5-hexen-1,2-diol; 5-hexen-1-ol; hydroxypropyl acrylate; 3-hydroxy-3,7,11-trimethyl-1,6,10-dodecatriene; isoamyl methacrylate; isobutyl methacrylate; isoprene; 2-isopropenylaniline; isopropenyl chloroformate; 4,4′-isopropylidene dimethacrylate; 3-isopropyl-a-a-dimethylbenzene isocyanate; isopulegol; itaconic acid; itaconalyl chloride; lead (II) acrylate; (±)-:linalool; linalyl acetate; p-mentha-1,8-diene; p-mentha-6,8-dien-2-ol; methyleneamino acetonitrile; methacrolein; [3-(methacryloylamino)-propyl]trimethylammonium chloride; methacrylamide; methacrylic acid; methacrylic anhydride; methacrylonitrile; methacryloyl chloride; 2-(methacryloyloxy)ethyl acetoacetate; (3-methacryloxypropyl)trimethoxy silane; 2-(methacryloxy)ethyl trimethyl ammonium methylsulfate; 2-methoxy propene (isopropenyl methyl ether); methyl-2-(bromomethyl)acrylate; 5-methyl-5-hexen-2-one; methyl methacrylate; N,N′-methylene bisacrylamide; 2-methylene glutaronitrite; 2-methylene-1,3-propanediol; 3-methyl-1,2-butadiene; 2-methyl-1-butene; 3-methyl-1-butene; 3-methyl-1-buten-1-ol; 2-methyl-1-buten-3-yne; 2-methyl-1,5-heptadiene; 2-methyl-1-heptene; 2-methyl-1-hexene; 3-methyl-1,3-pentadiene; 2-methyl-1,4-pentadiene; (±)-3-methyl-1-pentene; (±)-4-methyl-1-pentene; (±)-3-methyl-1-penten-3-ol; 2-methyl-1-pentene; alpha.-methyl styrene; t-a-methylstyrene; t-beta-methylstyrene; 3-methylstyrene; methyl vinyl ether; methyl vinyl ketone; methyl-2-vinyloxirane; 4-methylstyrene; methyl vinyl sulfone; 4-methyl-5-vinylthiazole; myrcene; t-beta-nitrostyrene; 3-nitrostyrene; 1-nonadecene; 1,8-nonadiene; 1-octadecene; 1,7-octadiene; 7-octene-1,2-diol; 1-octene; 1-octen-3-ol; 1-pentadecene; 1-pentene; 1-penten-3-ol; t-2,4-pentenoic acid; 1,3-pentadiene; 1,4-pentadiene; 1,4-pentadien-3-ol; 4-penten-1-ol; 4-penten-2-ol; 4-phenyl-1-butene; phenyl vinyl sulfide; phenyl vinyl sulfonate; 2-propene-1-sulfonic acid sodium salt; phenyl vinyl sulfoxide; 1-phenyl-1-(trimethylsiloxy)ethylene; propene; safrole; styrene (vinyl benzene); 4-styrene sulfonic acid sodium salt; styrene sulfonyl chloride; 3-sulfopropyl acrylate potassium salt; 3-sulfopropyl methacrylate sodium salt; tetrachloroethylene; tetracyano ethylene; tetramethyldivinyl siloxane; trans 3-chloroacrylic acid; 2-trifluoromethyl propene; 2-(trifluoromethyl)propenoic acid; 2,4,4′-trimethyl-1-pentene; 3,5-bis(trifluoromethyl)styrene; 2,3-bis(trimethylsiloxy)-1,3-butadiene; 1-undecene; vinyl acetate; vinyl acetic acid; 4-vinyl anisole; 9-vinyl anthracene; vinyl behenate; vinyl benzoate; vinyl benzyl acetate; vinyl benzyl alcohol; 3-vinyl benzyl chloride; 3-(vinyl benzyl)-2-chloroethyl sulfone; 4-(vinyl benzyl)-2-chloroethyl sulfone; N-(p-vinyl benzyl)-N,N′-dimethyl amine; 4-vinyl biphenyl (4-phenyl styrene); vinyl bromide; 2-vinyl butane; vinyl butyl ether; 9-vinyl carbazole; vinyl carbinol; vinyl cetyl ether; vinyl chloroacetate; vinyl chloroformate; vinyl crotanoate; vinyl cyclohexane; 4-vinyl-1-cyclohexene; 4-vinylcyclohexene dioxide; vinyl cyclopentene; vinyl dimethylchlorosilane; vinyl dimethylethoxysilane; vinyl diphenylphosphine; vinyl 2-ethyl hexanoate; vinyl 2-ethylhexyl ether; vinyl ether ketone; vinyl ethylene; vinyl ethylene iron tricarbonyl; vinyl ferrocene; vinyl formate; vinyl hexadecyl ether; vinylidene fluoride; 1-vinyl imidizole; vinyl iodide; vinyl laurate; vinyl magnesium bromide; vinyl mesitylene; vinyl 2-methoxy ethyl ether; vinyl methyl dichlorosilane; vinyl methyl ether; vinyl methyl ketone; 2-vinyl naphthalene; 5-vinyl-2-norbornene; vinyl pelargonate; vinyl phenyl acetate; vinyl phosphonic acid, bis(2-chloroethyl)ester; vinyl propionate; 4-vinyl pyridine; 2-vinyl pyridine; 1-vinyl-2-pyrrolidinone; 2-vinyl quinoline; 1-vinyl silatrane; vinyl sulfone; vinyl sulfone (divinylsulfone); vinyl sulfonic acid sodium salt; o-vinyl toluene; p-vinyl toluene; vinyl triacetoxysilane; vinyl tributyl tin; vinyl trichloride; vinyl trichlorosilane; vinyl trichlorosilane (trichlorovinylsilane); vinyl triethoxysilane; vinyl triethylsilane; vinyl trifluoroacetate; vinyl trimethoxy silane; vinyl trimethyl nonylether; vinyl trimethyl silane; vinyl triphenyphosphonium bromide (triphenyl vinyl phosphonium bromide); vinyl tris-(2-methoxyethoxy)silane; vinyl 2-valerate and the like.

Acrylate-terminated or otherwise unsaturated urethanes, carbonates, and epoxides can also be used in a polymer provided herein. An example of an unsaturated carbonate is allyl diglycol carbonate (CR-39). Unsaturated epoxides include, but are not limited to, glycidyl acrylate, glycidyl methacrylate, allyl glycidyl ether, and 1,2-epoxy-3-allyl propane.

In other embodiments, a polymer of the invention may be a “peptoid.” Generally speaking, a “peptoid” is a poly (N-substituted glycine), sharing the backbone structure of a protein, but having pendant groups (i.e., side chains) pendant from the amide nitrogens and that may be the same as, or differ in whole or in part from, conventional amino acid groups. The peptoid may be composed of monomers that have, for example, affinity property groups, hydrophilic groups, and/or hydrophobic groups pendant from their backbones. Peptoids are known in the art and have been described in various publications, including U.S. Pat. Nos. 5,877,278, 5,977,301, 5,831,005, 5,811,387 and 6,783,929, each of which is incorporated by reference herein for all purposes. Numerous diverse side chains are available for peptoid synthesis, including, for example, those derived from many natural or nor-amino acid, unnatural amino acid, nucleotide base, enzyme cofactor, or sugar. Further, side chains having isosteric and/or isoelectronic properties of known amino acid side chains and/or nucleotide bases, or surrogates thereof may be used. Examples of non-natural amino acid side chains are described in U.S. Pat. No. 5,656,660, incorporated by reference herein.

Other possible side chains may include, without limitation, those derived from the following: acetichydrazide, N-acetylethylenediamine, 1-adamantamine, 2-adamantamine-HCl, 1-adamantanemethylamine, alanine, beta-alanine, alaninamide, allylamine, O-allylhydroxylamine, 3-amino-1,2-propandiol, 2-amino-1,3-propanediol, 4-amino-1-benzylpiperidine, 2-amino-1-butanol, 4-amino-1-butanol, 6-amino-1-indanone, 5-amino-1-naphthol, 5-amino-1-pentanol, 2-amino-1-phenylethanol, 3-amino-1-propanol, 5-amino-2-methoxypyridine, 1-amino-2-propanol, aminoacetonitrile, 4-aminobenzamide, 4-aminobenzylcyanide, 4-aminobiphenyl, 4-aminobutyricacid, 6-aminocaproicacid, 1-aminocyclohexan-2,3-diol, 1-aminocyclohexan-3,4,5-triol, trans-4-aminocyclohexanol, aminodiphenylmethane, 3-amino-N′,N′-didecylpropanamide, 3-amino-N′,N′-dihexylpropanamide, 3-amino-N′,N′-dioctylpropanamide, 3-amino-N′,N′-diphenethylpropanamide, 2-aminoethanol, N-(aminoethyl)carbazole, 2-aminoethylmethacrylate, 4-(2-aminoethyl)morpholine, (2-aminoethyl)phenylamine, aminomethylphosphonate, 2-(2-aminoethyl)pyridine, aminoethyl-5-(2,3-dichlorothiophenyl)sulfonamide, N-aminoethylthymine, 2-(2-aminoethoxy)ethanol, 6-aminogalactose, 2-aminoheptane, 1-aminoindan, 2-aminoindan, 5-aminoindan, 5-aminoindole, 4-aminomethylbenzenesulfonicacid, 2-(aminomethyl)benzimidazole 4-(aminomethyl)benzoicacid, (aminomethyl)cyclohexane, trans-4-aminomethyl)cyclohexanecarboxylicacid, (aminomethyl)cyclopropane, 2-(aminomethyl)pyridine, 3-(aminomethyl)pyridine, 4-(aminomethyl)pyridine, 5-aminomethyl-2-naphthalenesulfonicacid, 2-amino-5-methyloctane, 2,2-aminomethylphenylthiobenzylalcohol, 1-aminonaphthalene, 2-aminonaphthalene, 1-(4-aminophenyl)-ethylamine, 4-aminophenylphenylether, 2-(4-aminophenyl)-ethylamine, 2,4-aminophenyl)ethylamine, 2-arninpropane-1,3-diol, 3-aminopropanol, 1-3-aminopropyl)-2-pyrrolidinone, 1-3-aminopropyl)imidazole, 4-(3-aminopropyl)morpholine, 3-aminopyridine, 4-aminostyrene, amylamine, aniline, arginine, asparticacid, benzenesulfonylhydrazide, 1,4-benzodioxan-6-amine, benzoichydrazide, benzylcarbazate, N-benzyl-2-phenethylamine, benzylarmine, O-benzylhydroxylamine, biphenylamine, 1,4-bis(3-aminopropyl)piperazine, 3,5-bis(trifluoromethyl)benzylamine, 3-butoxypropylamine, t-butylcarbazate, 4-t-butylcyclohexylamine, O-t-butylhydroxylamine, t-butylamine, butylamine, 4-butylaniline, 4-sec-butylaniline, 3-chloroaniline, 4-chloroaniline, 2-chloro-6-fluorobenzylthioethylamine, 2-(2-chlorophenyl)ethylamine, 2-(3-chlorophenyl)ethylamine, 2-(4-chlorophenyl)ethylamine, 5-chlorothiophene-2-sulphonylhydrazide, cycloheptylamine, 2-(1-cyclohexenyl)ethylamine, cyclohexylamine, 4-cyclohexylaniline, cyclopentylamine, cyclopropylamine, cysteamine, 2-(decyl)dodecylamine, decylamine, dehydroabietylamine, 1,4-diaminobutane, trans-1,4-diaminocyclohexane, N,N-di-(2-aminoethyl)amine, 1,5-diaminonaphthalene, 1,3-diaminopropan-2-ol, 1,3-diaminopropane, N,N-dibenzylglycinamide, 3,4-dichlorobenzylamine, N,N-dihexylglycinamide, 2,3-dihydroxypropylamine, 6,7-dimethoxy-1,2,3,4-tetrahydroisoquinoline, 4,4′dimethoxybenzhydrylglycinamide, 2,3-dimethoxybenzylamine, 3,5-dimethoxybenzylamine, 2,4-dimethoxybenzylamine, 3,4-dimethoxyphenethylamine, 3-dimethylaminopropylamine, 3,4-dimethylaniline, N,N-dimethylethylenediamine, 2,4-dimethylbutylamine, 3,7-dimethyloctylamine, 3,3-diphenyl-2-propenamine, 1,2-diphenylethylamine, 2,2-diphenylethylamine, 3,3-diphenylpropylamine, dodecylamine, epinephrine, ethanolamine, 3-ethoxypropylamine, 2-(4-ethoxy)phenethylamine, ethylamine, 4-ethylaniline, ethylenediamine, 2,2′-(ethylenedioxy)bis(ethylamine), 2-ethylhexylamine, 2-ethylpipecolinate, 1-ethylpropylamine, alpha-ethyltryptamine, 9-fluoreneamine, 4-fluorobenzylamine, N(2-fluorophenyl)piperazine, furfurylamine, D-glucosamine, glycinamide, glycine, glycine-beta-naphthylamide, guanidinoethylamine, guanidinopropylamine, guanidinobutylamine, 2-(4-guanidino)phenethylamine, 2,2,3,3,4heptafluorobutylamine, heptylamine, 1,6-hexanediamine, 1,6-hexanediamine, 3-hexenylamine, hexylamine, 2-(hexyl)octylamine, histamine, 3-hydroxytyrarmine, indoline, 4-iodoaniline, isoamylamine, isobutylamine, 3-isopropoxypropylamine, isopropylamine, 4-isopropylaniline, 4-methoxybenzenesulphonylhydrazide, 4-methoxybenzylcarbazate, 2-methoxybenzylamine, 3-methoxybenzylamine, 4-methoxybenzylamine, 2-methoxyethylamine, methoxylamine, 2-methoxyphenethylamine, 3-methoxyphenethylamine, 4-methoxyphenethylamine, 3-methoxypropylamine, methylhydrazine, methylhydrazineocarboxylate, N-methyl-2,2-diphenylethylamine, methylamine, alpha-(methylaminomethyl)-benzylalcohol, 4-methylbenzylamine, alpha-methylbenzylamine, 2-methylbutylamine, 3,4-(methylenedioxy)aniline, 3,4-methylenedioxyphenethylamine, beta-methylphenethylamine, 4 methylphenethylamine, cis-myritanylamine, 1(naphthyl)ethylamine, 1-naphthalenemethylamine, nicotinichydrazide, 4-nitrobenzoichydrazide, p-nitrophenethylamine, 3-nonenylamine, nonylamine, 2-norbornylamine, norephedrine, norphenylephrine, octopamine, octylamine, 2,2,3,3,3-pentafluoropropylamine, phenelzinesulfate, 2-phenethylamine, 2-(phenoxy)ethylamine, 3-phenoxy-2-hydroxypropylamine, 3-(phenyl)propargylamine, 1-phenyl-1,2,3,4-tetrahydroisoquinoline, 4-phenyl-1,2,3,4-tetrahydroisoquinoline, 3-phenyl-1-propylamine, 5-phenyl-O-anisidine, phenylacetichydrazide, phenylalanine, 4-phenylbutylamine, L-phenylephrine, 4-phenylsemicarbazide, 1-piperazinecarboxylate, piperidine, piperonylamine, propargylamine, propylamine, 4-propylaniline, serine, spermine, spernidine, 1,2,3,4-tetrahydro-1-naphthylamine, tetrahydrofurfurylamine, 1,2,3,4-tetrahydroisoquinoline, 2-thiophene-methylamine, p-toluenesulfonhydrazide, p-toluidine, 4-(trifluoromethyl)benzylamine, 3,4,5-trimethoxybenzylamine, 2,4,6-trimethoxybenzylamine, 2,4,6-trimethylbenzenesulfonylhydrazide, tryptamine, phospho-tyramine, tyramine, veratrylamine, m-xenylamine, m-xylylenediamine, and p-xylylenediamine.

The following documents, incorporated by reference herein, provide further examples of side chains that may be used: U.S. Pat. Nos. 5,877,278, 5,811,387, 5,447,916, 5,480,871, 5,919,967, and International Patent Application Nos. WO 91/19735, WO 96/40202, WO 96/40759, WO 97/19106, WO 94/03483, WO 95/04072, WO 98/09641, WO 98/52620, WO 97/10887 and WO 99/31124.

Side chains which may be used as affinity groups for synthesizing peptoids include alkyl, (cycloalkyl)alkyl, (cycloheteroalkyl)alkyl, aralkyl, and heteroaralkyl, each substituted optionally from oxo, thia, halo, amino, hydroxy, cyano, nitro, thio, aminocarbonyl, carboxy, and imino. The alkyl, (cycloalkyl)alkyl, (cycloheteroalkyl)alkyl groups may be further selected from methyl, hydroxymethyl, prop-2-yl, 2-methylpropyl, pyrrolidylmethyl, methylthioethyl, 1-hydroxyethyl, thioethyl, aminocarbonylmethyl, aminocarbonylethyl, carboxymethyl, carboxyethyl, 4-aminobutyl, and 3-guanidinopropyl, guanidinoaryl, hydroxyaryl, amidoalkyl, phosphonyl alkyl, phosphonyl aryl, oligoether, and polyhydroxyalkyl. The aralkyl and heteroaralkyl groups may be further selected from phenylalkyl, hydroxyphenylalkyl, imidazolylalkyl, purinylalkyl, pyrimidinylalkyl, and indolylalkyl. Examples of sugars include furanosylalkyl, pyranosylalkyl, furanosyl, or pyranosyl, attached at any suitable atom. Side chains which may be used for synthesizing peptoids in accordance with the present invention include those selected from the group of alkyloxyalkyl, hydroxyalkyl, thioalkyl, alkylthioalkyl, alkylsulfinylalkyl, alkyloxycarbonylalkyl, and aminocarbonylalkyl. The side chain may be further substituted, for example with one of methoxyethyl, hydroxyethyl, 1,3-dihydroxyprop-2-yl, 2-(hydroxymethyl)-1,3-dihydroxoxyprop-2-yl, and 2,3-dihydroxypropyl, alkylsulfoxidoalkyl, as well as side chains reported, or identified using the techniques described, in Chapman, R. G.; Ostuni, E.; Takayama, S.; Holmlin, R. E.; Yan, L.; Whitesides, G. M.; J. Am. Chem. Soc. 2000, 122, 8303-8304.

Also included are hybrid copolymers containing amino acid sequences derived from proteins found in structural biopolymers and synthetic non-peptide chains as described in Pub. No. US 2004/0102608, which is incorporated herein by reference. Hybrid copolymers also include, for example, a poly(N-substituted glycine) in which one or more internal or end residues are substituted with one of the 20 common amino acids in proteins, or with any other appropriate monomer.

Also included are “peptidomimetic” molecules. A peptidomimetic mimics the biological activity of a polypeptide but is no longer peptidic in chemical nature. A peptidomimetic is a molecule that contains no peptide bonds (that is, amide bonds between amino acids). However, the term peptidomimetic is sometimes used to describe molecules such as pseudo-peptides, semi-peptides and peptoids (discussed above).

Techniques of developing peptidomimetics from polypeptides are known. Further modifications can also be made by replacing chemical groups of the amino acids with other chemical groups of similar structure. The development of peptidomimetics can be aided by determining the tertiary structure of the original polypeptide, either free or bound to a ligand, by

NMR spectroscopy, crystallography and/or computer-aided molecular modeling. These techniques aid in the development of novel compositions of higher potency and/or greater bioavailability and/or greater stability than the original polypeptide [(Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98, all incorporated herein by reference].

In addition, polymers of the invention include peptides specifically selected for their ability to bind to a range of semiconductor surfaces with high specificity (see e.g., Whaley et al, Nature 405:665, 2000).

### Methods of Assembling Polymers

The chemical structure of a portion of the target analyte molecule (e.g., a “functionality”) can be used to intelligently design polymer(s) that bind to the functionality. The assembly approach to designing and synthesizing polymers and copolymers provides an enormous amount of flexibility in identifying synthetic polymers that bind to a target analyte molecule comprising specific functionalities. Libraries of such polymers may be synthesized and screened against a target analyte that includes the functionality, or an isolated functionality, in a process similar to the combinatorial screening process used by the pharmaceutical industry to identify lead compounds. Once identified, the polymer can be further refined to enhance its ability to bind to the chemical functionality associated with an analyte molecule of interest.

In one embodiment, a method for identifying a sequence specific polymer (SSP), is provided. The method includes providing a preselected target analyte molecule; providing a plurality of monomers; identifying monomers that individually, or in combination, interact with the target analyte or portion of the target analyte; assembling the monomers identified into at least one sequence specific polymer including a specific sequence and length of monomers, wherein the polymer comprises at least two monomers; contacting the sequence specific polymer with the preselected target analyte molecule; and identifying a sequence specific polymer that interacts with the preselected target analyte molecule. The sequence specific polymers can be sequentially presented to a GC/MS or other device to assess binding affinity with a target analyte, or as discussed in further detail below a plurality of sequence specific polymers can be examined on an addressable array.

Accordingly, a sequence specific polymer can be selected using a combinatorial screening method similar to those practiced in the pharmaceutical industry. Candidate monomers for incorporation into the polymer can be easily chosen based on the known characteristics of their chemical functionalities (e.g., hydroxyl, amino, nitro, sulfonate, phenyl, sulfhydryl, carboxyl, etc.). For example, nucleophilic functionalities in the polymer are expected to be attracted to electrophilic groups on the target. Through the application of standardized combinatorial-based screening methodologies, a library comprising a plurality of polymers with distinct sequences is generated, and the resulting library is screened against a target analyte molecule, or plurality of target analyte molecules. Usually, the analyte binding characteristics, such as the equilibrium binding constant or association and dissociation rate constants, are determined for individual members of the library, and computational techniques are employed to choose polymers with desirable values. Accordingly, the methods of the invention include the use of computer-based systems to assemble polymers of the invention. A “computer-based system” refers to the hardware means, software means, and data storage means used to analyze the information associated with monomer structure and target analyte structure. The minimum hardware of computer-based systems as they relate to the present invention include a central processing unit (CPU), input means, output means, and data storage means. A skilled artisan can readily appreciate that any one of the currently available computer-based systems is suitable for use in the present invention, including those which include a robotic system or component. The data storage means may include any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats may be used for storage, e.g. word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

In other embodiments, the structural characteristics of existing polypeptides can be used to design non-biopolymers of the invention. Nerve agents are known to bind specific enzymes/polypeptides and disrupt the ability of the enzyme to facilitate nerve function. By identifying the portion of the enzyme that is bound by the nerve agent, a series of polymers can be synthesized such that they share structural similarities with the region of the enzyme that interacts with the nerve agent. Consequently, the polymers may be screened to identify those that have an affinity for the nerve agent. Once identified, the polymers may be used in devices that detect the presence of the nerve agent under different environmental conditions.

A non-biopolymer can be assembled from a plurality of monomers to possess enhanced binding to a preselected target analyte molecule by exposing candidate polymers to the particular analyte or class of analytes that are being targeted for detection, and then selecting polymer(s) comprising a specific sequence of monomers that display enhanced binding for the target analyte. The term “enhanced binding” intends that the polymer bind more tightly to the preselected target analyte than to other types of molecules that would be considered “interfering” or environmental conditions that would be considered “interfering.” Accordingly, whether or not a specific sequence of monomers (i.e., a polymer) displays enhanced binding for a particular analyte may depend upon the environmental conditions under which the binding event takes place (e.g., temperature, relative humidity, pH, etc.). For example, a sequence may be selected for a high analyte dissociation temperature under elevated temperature conditions (e.g., 37° C., 50° C., 100° C., 150° C., 200° C., 250° C., or 300° C.). Further, a sequence may display enhanced binding under high pH (e.g., pH of 8, 9 or 100 or low pH (e.g., pH of 6, 5 or 4) conditions. A sequence specific polymer may have a K_{d} with its target analyte, for example, of 100 μM or lower, 10 μM or lower, 1 μM or lower, 100 nM or lower, 10 nM or lower, 1 nM or lower, or 100 pM or lower.

In addition, a sequence can be selected for low “interferent molecule” affinity. An “interferent molecule” is any molecule present in a sample comprising a target molecule that may bind to the sequence specific polymer and result in generation of a signal that cannot be distinguished from signal generated due to binding of the target molecule to the sequence specific polymer. Exemplary interferents include background components such as diatomic molecules (e.g., O₂ or N₂), triatomic molecules (e.g., H₂O or CO₂), and volatile or semivolatile chemical compounds. Accordingly, in another embodiment, a method of the invention includes providing a preselected target analyte molecule; providing a plurality of monomers; identifying monomers that individually, or in combination, interact with the target analyte or portion of the target analyte; assembling the monomers identified into at least one sequence specific polymer comprising a specific sequence and length of monomers, wherein the polymer comprises at least two monomers; contacting the sequence specific polymer with the preselected target analyte molecule; prior to, concurrent with, or subsequent to contacting the analyte, contacting the sequence specific polymer with a composition including an interferent; and identifying a sequence specific polymer and the preselected target analyte molecule.

A “sample,” as used herein relates to a material or mixture of materials containing or suspected of containing one or more target analytes of interest. A sample may include gaseous mediums, such as ambient air, chemical or industrial intermediates, chemical or industrial products, chemical or industrial byproducts, chemical or industrial waste, exhaled vapor, internal combustion engine exhaust, or headspace vapor such as vapor surrounding foods, beverages, cosmetics, vapor surrounding plant or animal tissue and vapor surrounding a microbial sample. Additional sample mediums include supercritical fluids such as supercritical CO₂ extractate. Other exemplary mediums include liquids such as water or aqueous solutions, oil or petroleum products, oil-water emulsions, liquid chemical or industrial intermediates, liquid chemical or industrial products, liquid chemical or industrial byproducts, and liquid chemical or industrial waste. Additional exemplary sample mediums include semisolid mediums such as animal or plant tissues, microbial samples, or samples containing gelatin, agar or polyacrylamide.

Referring again to FIG. 1, in one embodiment of the screening process, a plurality of SSPs (e.g., 2- or 3-mers) are first generated by synthesizing a library containing possible combinations of candidate monomers, and the analyte binding characteristics of each member of the library are evaluated to determine the most desirable subsets, or “best-in-class” members. Such libraries of SSPs can be generated on the basis of their expected binding affinities to chosen analytes by combining monomers into the SSPs that display or are expected to confer desirable binding affinities for the analyte of interest. The most desirable subsets are then used as building blocks to synthesize successively longer SSPs using the same sort of “best-in-class” analysis, until SSPs having optimal analyte binding characteristics are identified. The resulting polymers may be copolymers, multi-block polymers, and/or multimers of polymers, copolymers, or multi-block polymers. Optionally, successive rounds of screening can be used to optimize polymers identified as having an affinity for a chemical functionality, or multiple functionalities, associated with a target analyte molecule of interest.

Accordingly, a method for identifying a sequence specific polymer (SSP) includes providing a preselected target analyte molecule; providing a plurality of monomers; identifying a first set of monomers that individually, or in combination, interact with the target analyte or portion of the target analyte; assembling the monomers identified into a plurality of sequence specific polymers each comprising a specific sequence and length of monomers, wherein each polymer comprises at least two monomers; contacting the plurality of sequence specific polymers with the preselected target analyte molecule; detecting an interaction between the sequence specific polymers and the preselected target analyte molecule; selecting the sequence specific polymers that interact with the target analyte molecule; identifying a second set of monomers that individually, or in combination, interact with the target analyte or portion of the target analyte and adding the monomers to the polymers already identified; contacting the plurality of sequence specific polymers with the preselected target analyte molecule; and detecting an enhanced interaction between the sequence specific polymers and the preselected target analyte molecule.

In another embodiment, a method of the invention utilizes an array comprising sequence specific polymers to screen for those that possess enhanced binding of a target analyte under various conditions. Thus, methods provided herein include assembling, on an array, monomers to form a plurality of sequence specific polymers each comprising a specific sequence and length of monomers. In general, each polymer is associated with a specific address on the array.

Methods of the invention further include identifying a second set of monomers that individually, or in combination, interact with the target analyte or portion of the target analyte and adding the monomers to the polymers that are associated with an array. The monomers can be added to polymers at specific addresses on the array by techniques known to those skilled in the art of polymer and combinatorial chemistry. Subsequently, the plurality of sequence specific polymers can be re-screened with the preselected target analyte molecule.

The embodiments described herein are useful for detecting binding of a SSP to any molecular target, including a predetermined analyte. The term “analyte” or “analyte molecule,” also referred to herein as a “target analyte molecule,” encompasses a wide variety of substances and molecules, which range from simple molecules to complex targets found in gases, liquids, supercritical fluids, solids or semisolids. Target analyte molecules include any molecule that can interact with a polymer of the invention, such as molecules containing two or more atoms, three or more atoms, volatile or semivolatile chemical compounds, combustion products, metabolites, suspended particles, components of pesticides, components of an explosive device, components of chemical warfare agents, pharmaceutical agents, and positive or negative ions, either as individual particles or attached to a molecule. Accordingly, a target analyte molecule can include a chemical compound (i.e., non-biological compound such as, e.g., an organic molecule, an inorganic molecule, or a molecule having both organic and inorganic atoms), a mixture of chemical compounds, an array of spatially localized compounds, a biological macromolecule, a bacteriophage peptide display library, a polysome peptide display library, an extract made from biological materials such as bacteria, plants, fungi, or animal (e.g., mammalian) cells or tissue, a protein, a toxin, a peptide hormone, a cell, a virus, or the like.

Exemplary target analytes also include components of explosive materials. Examples of such materials include, but are not limited to, RDX, TNT, PETN, and EGDN. Exemplary analytes also include components of nerve agents. Examples of such agents include, but are not limited to, VX, sarin (GB) and tabun. Accordingly, a target analyte includes target chemical functionalities that provide binding sites for polymers provided herein. A target analyte includes explosive chemicals such as 2,4,6-trinitrotoluene (TNT) and 1,3,5-trinitrobenzene (TNB). For example, a chemical functionality on TNT may interact with a complementary portion of a functional monomer included in a polymer, either covalently or by other interactions such as ionic, hydrophobic or hydrogen bonding.

Additional target analytes include environmental pollutants (including heavy metals, organometallic compounds, pesticides, insecticides, toxins, etc.); chemicals (including solvents, polymers, organic materials, etc.); soluble, volatile and semivolatile metabolites found in foods or beverages, therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc.) (detection of antigen antibody interactions are described in U.S. Pat. Nos. 4,236,893, 4,242,096, and 4,314,821, all of which are expressly incorporated herein by reference); whole cells (including procaryotic (such as pathogenic bacteria) and eukaryotic cells, including mammalian tumor cells); viruses (including retroviruses, herpesviruses, adenoviruses, lentiviruses, etc.); and spores; etc.

Whether or not a sequence specific polymer of the invention displays enhanced binding to a target molecule may depend upon the various attributes inherent in the chemical structure of a target analyte. Further, it should be understood that as used herein, the term “target” refers not only to known, pre-selected analytes, but also to unknown targets in a sample that may bind to a sequence specific polymer of the invention. For example, a sample may contain an unknown target that includes a component or functional group that is recognized by a sequence specific polymer of the invention. Accordingly, the target can be any molecular structure, whether singular or part of a larger macromolecular structure. Additionally, the target can be referred to as a “preselected analyte type”, which includes the situation where the target is a single molecular species or a molecular moiety that is shared among members of a class or type of target molecules.

By way of example, the target may be a nucleic acid, which intends any polymeric nucleotide (i.e. “oligonucleotide” or “polynucleotide”), which in the intact natural state can have about 10 to 500,000 or more nucleotides and in an isolated state can have about 20 to 100,000 or more nucleotides, usually about 100 to 20,000 nucleotides, and more frequently 500 to 10,000 nucleotides. For example, the assay can be adapted to detect any target nucleic acid with a determined nucleic acid sequence that is characteristic of a cell type, cell morphology, pathology, bacteria, microbe, virus, etc.

The nucleic acid targets include nucleic acids from any source in purified or unpurified form including DNA (dsDNA and ssDNA) and RNA, including t-RNA, m-RNA, r-RNA, mitochondrial DNA and RNA, chloroplast DNA and RNA, DNA-RNA hybrids, or mixtures thereof, genes, chromosomes, plasmids, the genomes of biological material such as microorganisms, e.g., bacteria, yeasts, viruses, viroids, molds, fungi, plants, animals, humans, and fragments thereof, and the like. In one embodiment, the target is a double stranded DNA (dsDNA) or a single stranded DNA (ssDNA). The target can be obtained from various biological material by procedures well known in the art.

The target may also be recognizable by an antibody, in which case the target is any epitope or antigen, or any immunoreactive molecule, including antigen fragments, antibodies and antibody fragments (to which anti-immunoglobulin antibodies bind), both monoclonal and polyclonal, and complexes thereof, including those formed by recombinant DNA molecules. The term “hapten”, as used herein, refers to a partial antigen or non-protein binding member which is capable of binding to an antibody, but which is not capable of eliciting antibody formation unless coupled to a carrier protein.

### Combinatorial Libraries of Polymers

The methods described above are particularly useful for generating libraries of sequence specific polymers that display enhanced binding for a target analyte, or functional group included therein. As previously noted, the term “enhanced binding” intends that the polymer bind more tightly to the preselected target analyte than to other types of molecules that would be considered “interfering” or environmental conditions that would be considered “interfering.” Accordingly, methods described throughout the specification are suitable for generating libraries of polymers that display enhanced binding for a particular analyte under particular environmental conditions (e.g., temperature, relative humidity, pH, etc.). For example, a library of polymers may include members that have an affinity for a target analyte under elevated temperature conditions. Multiple libraries and sub-libraries can be manufactured by such methods.

Additional libraries and sub-libraries can be manufactured based upon the members' low “interferent molecule” affinity. As described above, an “interferent molecule” is any molecule present in a sample comprising a target molecule that may bind to the sequence specific polymer and result in generation of a signal that cannot be distinguished from signal generated due to binding of the target molecule to the sequence specific polymer. Exemplary interferents include background components such as diatomic molecules (e.g., O₂ or N₂), triatomic molecules (e.g., H₂O or CO₂), and volatile or semivolatile chemical compounds.

Accordingly, in another embodiment, a combinatorial library of sequence specific polymers is provided. The library may include polymers that bind to a particular analyte, or functional group thereof, but individual members of the library may have optimal affinity for the analyte under different environmental conditions or in the presence of different interferents. Thus, the present methods encompass generating libraries of polymers that possess affinity for an analyte and generating sub-libraries of polymers that have an affinity for the analyte under different conditions.

### Devices and Detection Methods

A detection device of the invention comprises a sequence specific polymer and a response transduction medium. In one embodiment, the device has the characteristic that binding of an analyte to the sequence specific polymer causes a change in a property of the sequence specific polymer that is transduced to the response transduction medium and results in a detectable signal.

In another embodiment, the device has the characteristic that the sequence specific polymer acts as a “concentrator component.” As a result, binding of an analyte to the sequence specific polymer does not cause a change in a property of the sequence specific polymer which is transduced to the response transduction medium. Rather, the high local concentration of analyte molecules provided by interaction with the sequence specific polymer causes a change in a property of the response transduction medium which results in a detectable signal In yet another embodiment of the invention, binding of an analyte to the sequence specific polymer causes both: 1) a change in a property of the sequence specific polymer that is transduced to the response transduction medium and results in a detectable signal, and 2) a change in a property of the response transduction medium by the analyte due to the “concentrator effect” provided by interaction of the analyte with the sequence specific polymer which then causes a detectable signal.

Other ingredients may be included with the sequence specific polymer which help to facilitate a transducible response. The sequence specific polymer may be admixed or copolymerized with such ingredients. The other ingredients include monomeric compounds such as electrochemically or redox active compounds, and optically absorbing or emitting compounds. The other ingredients also include nanoparticles such as carbon nanotubes, graphite or quantum dots. Additional other ingredients include synthetic or natural polymers such as electrochemically or other redox active polymers, electrically conductive polymers, and optically absorbing or emitting polymers.

A composition containing a sequence specific polymer and other ingredients include those in the form of a solid, semisolid or high viscosity liquid. Solid compositions include porous solids such as patterned ceramic, aerogel, zeolite or controlled-pore glass, and a colloidal solid. Semisolid compositions include colloidal suspensions and hydrogels.

In one embodiment of the invention, the detection device comprises a sequence specific polymer in a composition with other ingredients as set forth above, and a response transduction medium. Binding of an analyte to the sequence specific polymer does not cause a change in a property of the polymer which is transduced to the transduction medium. Instead the sequence specific polymer acts as a “concentrator component” such that binding of an analyte to the sequence specific polymer provides a high local concentration of analyte molecules which causes a change in a property of one or more of the other ingredients in the sequence specific polymer composition which is transduced to the response transduction medium, and then results in a detectable signal In yet another embodiment of the invention, binding of an analyte to the sequence specific polymer in the sequence specific polymer composition causes two or more of: 1) a change in a property of the sequence specific polymer that is transduced to the response transduction medium and results in a detectable signal, 2) a change in a property of one or more of the other ingredients in the sequence specific polymer composition due to the “concentrator effect” provided by interaction of the analyte with the sequence specific polymer, the change in property being transduced to the response transduction medium which then causes a detectable signal, and 3) a change in a property of the response transduction medium caused by the analyte due to the “concentrator effect” provided by interaction of the analyte with the sequence specific polymer which then causes a detectable signal.

Properties of the sequence specific polymer, other ingredients in the sequence specific polymer composition, or the response transduction medium which can be changed and that result in transduction of a response to the response transduction medium include chemical, thermodynamic, mechanical, thermal, electromagnetic and quantum mechanical properties. Exemplary chemical properties of the sequence specific polymer, other ingredients in the sequence specific polymer composition, or the response transduction medium which can be changed upon binding of an analyte include chemical bond connectivity, stereochemical configuration, conformation, ionization state, oxidation state or redox potential, and protonation state or pKa. Thermodynamic properties which can be changed include temperature, pressure, internal energy, volume, entropy, heat capacity, compressibility, electrochemical potential, and Gibbs and Helmholtz free energy. Mechanical properties which can be changed include dimension(s), mass density, intramolecular and intermolecular interaction forces, stiffness modulus, strength, fracture toughness, acoustic impedance, and the speed of sound. Thermal properties which can be changed include thermal conductivity and thermal diffusivity.

Exemplary electromagnetic properties of the sequence specific polymer, other ingredients in the sequence specific polymer composition, or the response transduction medium which can be changed upon binding of an analyte include work function, real and imaginary linear susceptibilities and permeabilities at one or more oscillation frequencies including zero (dc), real and imaginary nonlinear susceptibilities and permeabilities at one or more oscillation frequencies including zero (dc), real and imaginary linear permittivity at one or more oscillation frequencies including zero (dc), real and imaginary nonlinear permittivity at one or more oscillation frequencies including zero (dc), charge density, and charge mobility at one or more frequencies including zero (dc). Quantum mechanical properties which can be changed include energies of quantum states, wavefunction amplitude and distribution of quantum states, transitions between quantum states, and transition rates between quantum states.

The detectable signal which results from transduction of the change in property of the sequence specific polymer, other ingredients in the sequence specific polymer composition, or the response transduction medium may be proportional to the concentration of the analyte, or it may be a binary signal (yes versus no response) with transition occurring at a certain analyte concentration or over a narrow range of analyte concentrations. The detectable signal includes an electrical (e.g., capacitance), mechanical, optical, acoustic or thermal signal. The detectable signal can also be actuation of a process, such as change in the delivery rate of a drug or actuation of a valve. The detectable signal can also be deflection of a deflectable element, such as a membrane or cantilever.

The detection device of the invention includes embodiments in which the sequence specific polymer is present at a liquid-gas or liquid-liquid interface. Examples of such interfaces include water-air or water-oil interfaces. Other embodiments include those in which the sequence specific polymer is on a solid or semisolid surface. Examples of such surfaces include metal, glass, ceramic, fabric, natural and non-natural polymers, and hydrogels.

The sequence specific polymer, or composition containing the sequence specific polymer and other ingredients, is associated with a response transduction medium in the detection device of the invention. In some embodiments, the sequence specific polymer or composition containing the sequence specific polymer is placed on, affixed to, or otherwise operably associated with a response transduction medium. In other embodiments, the sequence specific polymer or composition containing the sequence specific polymer is mixed or otherwise integrally associated with the response transduction medium. In yet other embodiments, the sequence specific polymer or composition containing the sequence specific polymer is covalently or otherwise attached to form an integral molecular entity with the response transduction medium. In another embodiment, the response transduction medium, such as a deflectable element, may provide structural features that allow a plurality of sequence specific polymers to be positioned or arrayed in a pattern such that each polymer is assigned a specific address in or on the response transduction medium.

For example, the sequence specific polymers of the present invention may be incorporated into a molecular monolayer attached by physisorption or chemisorption onto a solid or semisolid surface of the response transduction medium, and thereafter exposed to a sample medium. The solid surface may be a metal, glass, ceramic, a polymer, a hydrogel, or any combination of these materials. In addition, the sequence specific polymers may be crosslinked with each other to form a polymer matrix of pure sequence specific polymer.

In one embodiment, a detection method of the invention comprises exposing a sample to a sequence specific polymer under conditions such that an analyte in the sample binds to the sequence specific polymer. Binding of the analyte to the specific polymer results in a change in a property of the sequence specific polymer that is transduced to a response transduction medium which generates a detectable response, and the response is then detected. In other aspects of the method, there is no change in properties of the sequence specific polymer upon binding of analyte. Instead the “concentrator effect” provided by binding of the analyte to the sequence specific polymer provides for change in a property of an other ingredient in a sequence specific polymer composition which is transduced to the response transduction medium, or the “concentrator effect” causes a change in a property of the response transduction medium. In yet other aspects of the invention, the detection method may involve two or more of: 1) a change in a property of the sequence specific polymer that is transduced to the response transduction medium and results in a detectable signal, 2) a change in a property of one or more of the other ingredients in the sequence specific polymer composition due to the “concentrator effect” provided by interaction of the analyte with the sequence specific polymer, the change in property being transduced to the response transduction medium which then causes a detectable signal, and 3) a change in a property of the response transduction medium caused by the analyte due to the “concentrator effect” provided by interaction of the analyte with the sequence specific polymer which then causes a detectable signal.

In one embodiment of the invention, the sequence specific polymer or composition containing the sequence specific polymer can be on a solid surface which acts as the response transduction medium. The solid surface includes, for example, a deflectable element such as a membrane or a cantilever. In certain embodiments binding of the target analyte to the sequence specific polymer can result in a conformational change of the polymer which in turn causes deflection of the membrane or cantilever, which in turn is monitored by an appropriate sensor.

In another embodiment the sequence specific polymer or composition containing the sequence specific polymer can be incorporated into, or placed upon, an electrically conductive element or a fluorescent medium which acts as the response transduction medium. A property change in the sequence specific polymer upon binding of an analyte is transduced to the electrically conductive element or fluorescent medium which then results in a signal that is monitored by an appropriate sensor.

Another embodiment of the invention involves associating the sequence specific polymer or composition containing the sequence specific polymer with actuation of a process that acts as the response transduction medium and “signal generator.” For example, a property change in the sequence specific polymer upon binding of an analyte can be transduced so as to change the delivery rate of a drug or actuate a valve.

In yet another embodiment, the sequence specific polymer or composition containing the sequence specific polymer is in association with a medium, such as a gaseous medium, and induction of binding and nucleation of condensed matter acts as the response transduction medium. Thus, upon binding of an analyte to the sequence specific polymer, a property change occurs which induces binding and nucleation of condensed matter in the medium, such as a gaseous medium, which is monitored by an appropriate sensor.

Another embodiment involves the covalent linkage of a sequence specific polymer to a light generating protein which acts as the transduction response medium. For example, the sequence specific polymer can be covalently joined directly or by a linker to the C-terminus, the N-terminus, or a pendant functional group of the luciferase protein. Upon binding of an analyte to the sequence specific polymer, a property change occurs which induces light generation by the luciferase protein which is monitored by an appropriate sensor.

In other embodiments, a detection device of the subject invention includes a response transduction medium that comprises an array which includes sequence specific polymers, and methods of manufacturing such response transduction medium arrays. Specifically, the subject invention provides methods for fabricating an array that enables the precise control over the length and sequence of polymers associated with the array. Accordingly, an array of the invention provides a mechanism for assembling, screening and identifying polymers for enhanced binding to a target analyte. Such arrays are useful in the process of manufacturing polymers of the invention.

In some embodiments, a response transduction medium array of the invention includes a plurality of polymers that have been identified as possessing enhanced binding to a target analyte. Such arrays are useful in devices that detect the presence or absence or quantity of the target analyte in a sample. The arrays of the subject invention may be employed in array assays in which the arrays are contacted with a sample containing, or suspected of containing, one or more target analytes of interest. Once contacted, and further processed if required, any polymer/target binding complexes present on the array may be detected to provide information about the presence of the one or more targets in the sample.

A response transduction medium array of the invention may include, for example a plurality of sequence specific polymers, each with an enhanced binding specificity for a different target analyte. Thus, multiple analytes can be monitored in a single sample. In another aspect, a plurality of different sequence specific polymers is provided on a response transduction medium, but each sequence specific polymer is designed to have an enhanced binding specificity for the same target analyte. While, in theory, it is possible to design a sequence specific polymer with absolute, or near absolute, specificity for a single target analyte, in practice sequence specific polymers will often exhibit some cross reactivity with molecules which are not the desired target analyte. Therefore, by using an array with multiple different sequence specific polymers with enhanced binding affinity for a desired target analyte, there is an increased likelihood that a positive response from all of the sequence specific polymers will correlate with the detection of a desired target analyte. A response by some, but not all, of the sequence specific polymers would be indicative that a molecule other than the target analyte was being detected, such as an interferent or some molecule with a close structural relationship to the target analyte. In yet another aspect of the invention, multiple sequence specific polymers designed to bind a desired analyte may be associated with separate detection devices and the collective responses of all the devices can be used to assess the amount of a target analyte in a sample.

An array includes any one, two or three-dimensional arrangement of addressable regions bearing a particular chemical moiety or moieties (for example, sequence specific polymers) associated with that region. Each region may extend into a third dimension in the case where the substrate or response transduction medium is porous while not having any substantial third dimension measurement (thickness) in the case where the substrate or response transduction medium is non-porous. An array is “addressable” in that it has multiple regions of different moieties (for example, different sequence specific polymers) such that a region (a “feature” or “spot” of the array) at a particular predetermined location (an “address”) on the array will detect a particular target analyte or class of targets that share a common functionality. Any given substrate may carry one, two, four or more arrays disposed on a surface of the substrate. Depending upon the use, any or all of the arrays may be the same or different from one another and each may contain multiple spots or features.

Accordingly, an array may contain one or more, including more than two, more than ten, more than one hundred, more than one thousand, more than ten thousand features, or even more than one hundred thousand features, in an area of less than 20 cm² or even less than 10 cm², e.g., less than about 5 cm², including less than about 1 cm², less than about 1 mm², e.g., 100 μm²; or even smaller. In certain embodiments, an array may cover an area as great as about 230 cm² or more, e.g., as great as about 930 cm² or more.

An “array assembly” may be one or more arrays plus a substrate on which the one or more arrays are deposited, although the assembly may be in the form of a package which includes other elements (such as a housing with a chamber). There are two main ways of producing polymer arrays, i.e., via in-situ synthesis in which the polymer is assembled on the surface of the substrate in a step-wise fashion and via deposition of the full polymer, e.g., a pre-synthesized sequence specific polymer, onto, into, or within the various locations of the array. It is understood that the present invention encompasses these processes.

Embodiments of the subject invention enable an array to be prepared or “customized” at least with respect to each feature size of the prepared array. This customization may be accomplished by determining an array layout in which each feature in the array layout has a size that is chosen based on its composition, and fabricating an array according to the polymer array layout.

In general, an array of the invention comprises a suitable substrate (e.g., a response transduction medium) and a plurality of sequence specific polymers. By “substrate” or “solid support” or other grammatical equivalents, herein is meant any material appropriate for the attachment of polymers and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, ceramics, and a variety of other polymers.

In yet another embodiment, a detection device that includes a sequence specific polymer and a response transduction medium is provided which further includes a microsensor operably associated with the device. In some aspects, the microsensor detects a change in a mechanical, chemical, optical, photonic, electrical, thermal or magnetic property that is modified by the binding event between a sequence specific polymer and a target analyte. In some aspects, the device includes an array. In other aspects, the microsensor is a deflectable member such as a membrane or a cantilever.

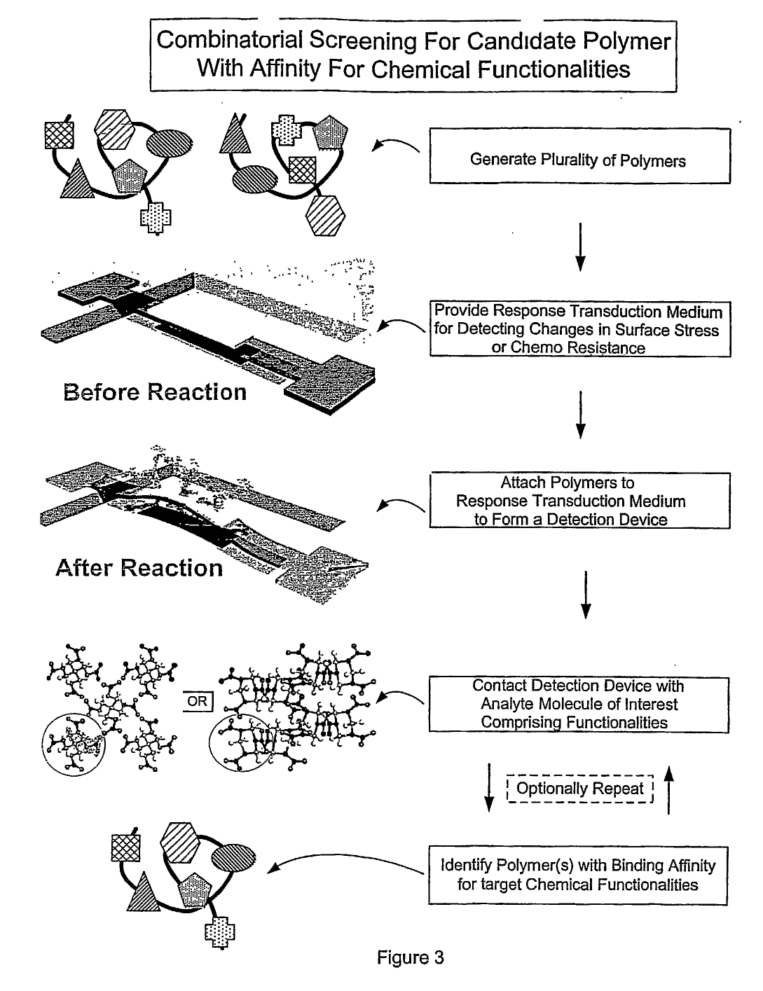

Referring to FIG. 3, a device of the present invention employs sequence specific polymers as a molecular detection moiety. The sequence specific polymers may be arranged in an array such that each SSP is associated with a particular response transduction medium, thereby resulting in an array of “tranducer elements” formed from each set of SSP/response transduction medium pairs. Sequence specific polymers of the invention are designed to exhibit a specific binding affinity for a predetermined target, which may be a single molecular species or a related family of molecular species. The target may be present in a sample that is in the gas, vapor, liquid and/or hydrogel (i.e. semisolid) state. The polymer may be attached to a stress- or chemo-sensitive response transduction medium that is modified when the polymer binds to a target analyte. A signal is generated such that the binding event is detected. The signal may take the form any detectable chemical, mechanical, thermodynamic, thermal, electromagnetic, or quantum mechanical signal generated by the interaction of a sequence specific polymer and target analyte. In the event of specific binding between a polymer and an analyte, the signal may be generated by the polymer, the target analyte, the target-analyte combination, by an intermediary molecule that is part of a composition associated with the polymer, or by a component of the response transduction medium.

Accordingly, the micromechanical sensors provided herein include microsensors such as micromechanical surface stress sensors (MSSS). Terms such as “deformation detector” or “deflection sensor” are used herein to denote conventional electrical and/or optical circuit elements designed to detect changes in conformation or configuration of a structure such as a micro-cantilever. The “deformation detector” or “deflection sensor” may optionally measure the degree of the deformation or change in configuration. Such detectors or sensors may be resistive, optical or resonant. In the case of a resistive-type detector or sensor, piezoelectric circuit elements incorporated into the micro-response transduction medium, such as a micro-cantilever, are sensitive to changes in dimension. In the case of optical monitoring device, a laser beam is aimed at the subject micro-response transduction medium, a reflected beam being monitored by an optical sensor. Motion of the point of incidence of the reflected beam on the sensor is a measure of the movement of the micro-response transduction medium. Where resonance is used to monitor micro-response transduction medium deformation, a circuit element on the micro-response transduction medium is included in a resonant circuit. A deformation of the micro-response transduction medium changes the resonant frequency. All of these measurement techniques are well-known in the art and can be implemented without additional explanation.

The term “cantilever” or “microcantilever” is used herein to denote any structural element that is so anchored as to have at least one degree of freedom, enabling movement in at least one dimension. The movement is usually a bending, rotational and/or torsional motion. In one embodiment, the movement occurs in response to a binding event between a sequence specific polymer and a target analyte provided on at least one surface of the cantilever or microcantilever. A cantilever or microcantilever generally has one end fixed to a substrate and an opposite end which is free and unattached. For example, a cantilever or microcantilever may take the form of a planar finger-like projection extending from a base or substrate into a space such as a gas- or liquid-containing chamber. Generally, microcantilevers are made of a semiconductor material. However other materials may be used, provided that such materials are capable of being fabricated in the requisite size, for instance, by a mask aligner. Microcantilevers are of a microscopic size, with a thickness on the order of 1 μm (e.g., 800 nm), a width on the order of 10 μm (e.g. 30 μm), and a length on the order of 100 μm (e.g., 200 or 300 μm). By “micro-membrane” is meant a thin disk or other shape preferably pre-coated with a wide range of films selected from metals, polymers, ceramics to bio-molecules. The micro-membrane may be oscillated at its resonance frequency. A large number of different micromembranes exist, see for example E. Quandt, K. Seemann, Magnetostrictive Thin Film Microflow Devices, Micro System Technologies 96, pp. 451-456, VDE-Verlag GmbH, 1996, which is expressly incorporated herein by reference.

In general, a change in a mechanical property of a microcantilever or micromembrane can, for example, be stress formation in the microcantilever or micromembrane due to changes in surface tension of the microcantilever or micromembrane. Stress formation can also occur due to changes in temperature of the microcantilever or micromembrane due to a bimorph effect, if the microcantilever or micromembrane is made of two materials with different thermal expansion coefficients. Such stress often results in the deflection or bending of the microcantilever or micromembrane. Stress can also be the result of an increase or decrease on the mass of the microcantilever or micromembrane which can result in deflection of the microcantilever or micromembrane. Such stress or deflection in the micro-cantilever can be detected in a variety of ways. If deflection of the microcantilever or micromembrane occurs, the deflection can be detected for example by a laser beam, a reflecting surface of the micro-cantilever and an optical detector to measure the deflection angle.

An alternative method of detecting changes on a microcantilever or micromembrane is detection of changes in an electrical property of a piezoelectric element integrated with the microcantilever or micromembrane. This method has an advantage in that it does not require optical access to the microcantilever or micromembrane. In general, at least one electrical parameter of the piezoelectric element is measured to detect a change in the microcantilever or micromembrane. Such parameters include resistance, current or voltage.

Additionally or alternatively, changes in resonance frequency or amplitude can be used to detect a change in a mechanical property of the microcantilever or micromembrane.

A change of mass of the microcantilever or micromembrane occurs when sufficient material binds to the microcantilever or micromembrane, so as to produce a change in the resonance frequency or amplitude of vibration of the microcantilever or micromembrane. Such changes can be monitored by use of an oscillator to vibrate the microcantilever or micromembrane at or near a frequency near its resonance frequency. Changes in the amplitude or resonant frequency of the dynamic bending of the microcantilever or micromembrane can be measured using the piezoelectric element and measuring one or more electrical parameters. Alternatively, a laser or other source of radiation may be used to detect the sequence frequency and/or amplification of vibration of the microcantilever or micromembrane.

The use of micromechanical devices is advantageous in methods provided herein for several reasons. Various signals such as force, heat, stress, magnetism, charge, radiation and chemical reactions can be readily transduced into a micromechanical deflection by an appropriately coated structure, such as a cantilever. In addition, silicon-based micromechanical devices can easily be integrated into microelectronic processing systems such as CMOS (Complementary Metal-Oxide-Semiconductor), as known to one of skill in the art. As a result, it is possible to produce seamless devices as low cost and to integrate them with other electronic devices such as computers. Moreover, micromechanical devices are very small, typically approximately 400 μm in length, approximately 40 μm wide and approximately 1 μm thick. As a result, it is possible to obtain a short response time, generally measured in microseconds, as well as sensitivity superior to standard techniques. Finally, it is possible to construct arrays of micromechanical devices, thereby permitting complex analysis of a variety of signals as well as the use of a variety of sensing materials.

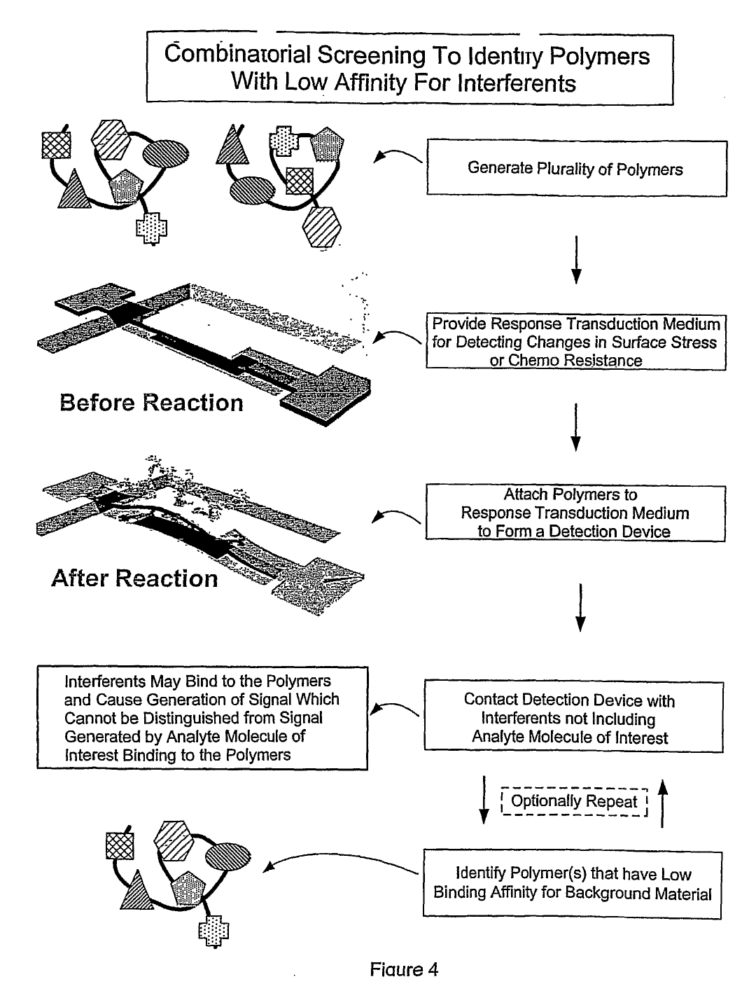

Referring to FIG. 4, methods and systems of the invention can be used to screen for sequence specific polymers that do not bind to the materials that comprise a target analyte of interest (i.e., “background material”). For example, the micro-response transduction media used in the aforementioned devices include micro-cantilevers and micromembranes that may be treated with a sequence specific polymer that specifically binds to a target analyte. These polymers may then be exposed to materials that do not contain the target analyte. Those polymers that do not bind to, or have low affinity for, such materials are chosen for further use because they are unlikely to give “false positives” in the presence of non-target containing materials. For example, sequence specific polymers that specifically bind to a target analyte component of an explosive material can be screened against those components of the explosive material that do not contain the target analyte. Those polymers identified as having high affinity for the target analyte, and low affinity for the materials that do not contain the target analyte, are selected because they are less likely to provide false positive signals.

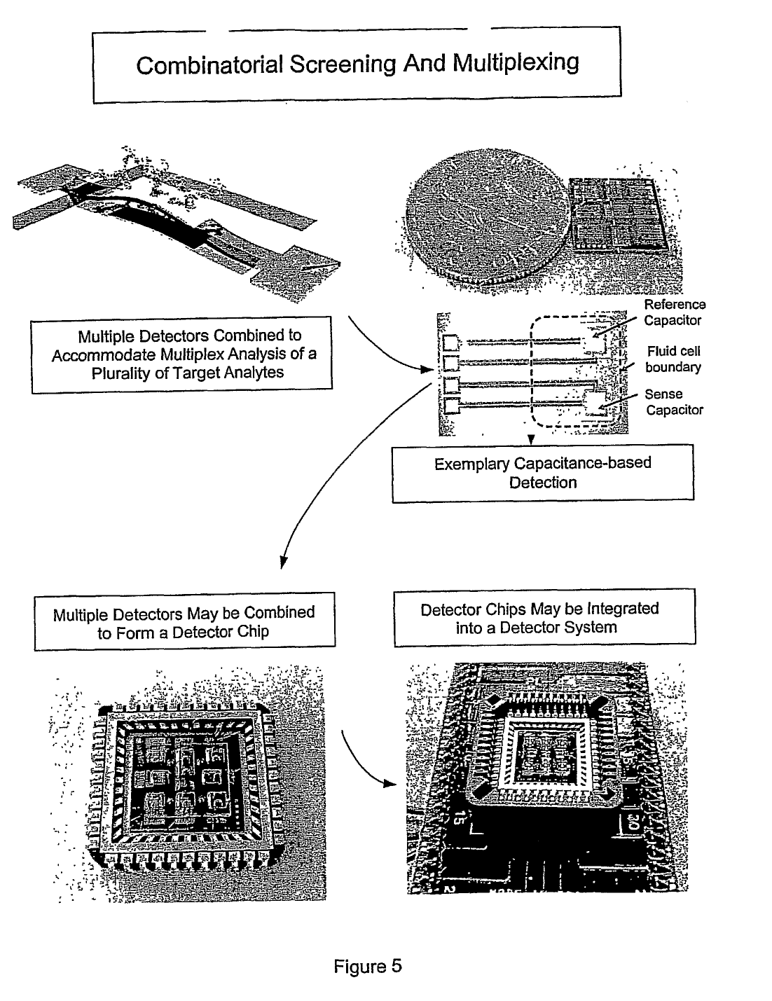

Referring to FIG. 5, an apparatus of the invention may also include a plurality of cantilevers or membranes for the detection of a plurality of target analytes in semi-solid or vapor phases. Preferably, the cantilevers or membranes of the invention are positioned in a channel or chamber. The channel or chamber has inlet or outlet ports which allow for the introduction of samples into the channel or chamber for analysis of target samples. In one embodiment, the sample may be separated, for example, into different channels or chambers for separate analysis. That is, in one embodiment multiple samples can be analyzed simultaneously. In an alternative embodiment multiple target analytes can be analyzed from a single sample. That is, a plurality of discrete microdevices may be contained within a single chamber. In this embodiment the individual microdevices may be used to detect discrete target analytes from a single sample.

With regard to detection devices of the invention, the sequence specific polymers of the present invention may be incorporated into detection systems involving the use of cantilevers, actuators, etc., including chemo-mechanical sensors (cantilever, membrane, etc.), quartz crystal microbalances, surface acoustic wave sensors, mechanical resonance sensors, chemFET sensors, chemically-sensitive “chemo-mechanical” valves for implantation into the body, e.g., insulin or other drug delivery

In other embodiments, methods are provided for identifying sequence specific polymers that bind to a target analyte specific to a particular disease, such as cancer. It is known that certain cell markers, such as antigenic determinants, are associated with neoplastic disorders. Usually these markers are only detectable once the disease has reached an advanced stage. The present invention provides the means to identify sequence specific polymers that are sensitive enough to detect the marker at an earlier point in the progression of the disease. For example, sequence specific polymers may be synthesized and screened for affinity to antigenic determinants known to be associated with lung cancer. Once identified, the candidate sequence specific polymers may be screened against the vaporous materials normally exhaled from a healthy lung. The candidate polymers that display the least amount of binding to these background materials can be identified. Once identified, these sequence specific polymers can be included in a sensor attached to a device suitable for detecting cell markers associated with the presence of lung cancer in a subject's exhalent. In another aspect, volatile exhalents not related to cell-surface markers, but which nevertheless are indicative of the presence of a tumor or other cancer, are monitored.

In other embodiments, methods are provided for using sequence specific polymers to develop “fingerprint” responses for different patient classes. For example, a detection device containing an array of different sequence specific polymers and a response transduction medium may be used to provide a response pattern for a sample obtained from a healthy patient, and also to provide a response pattern for a sample obtained from a patient in a disease class, such as cancer, diabetes, arthritis, etc. In another aspect, multiple detection devices with single sequence specific polymers and a transduction medium are used. The sample may be lung exhalant, urine, blood or any other biological sample. The binding specificity of the sequence specific polymers need not be known. However, having a large number of different sequence specific polymers with a diverse pattern of binding specificities will provide a more robust response pattern. A characteristic “fingerprint” response pattern for a healthy patient can be developed, which can then be compared to the characteristic “fingerprint” response pattern for a patient from various disease classes, thus providing for a method of disease diagnosis.

In other embodiments, systems are provided that are capable of detecting analytes in non-solid samples, i.e. samples that are not in the solid state, such as the gaseous, vapor, liquid, hydrogel or semi-solid state. Accordingly, the SSPs of the present invention are useful in detecting analytes in a wider variety of sample media than most conventional aqueous-based assays.

For example, the target analyte may be present in an industrial or clinical “test sample”, which includes biological samples that can be tested by the methods of the present invention described herein and include human and animal body fluids such as whole blood, serum, plasma, cerebrospinal fluid, urine, lymph fluids, and various external secretions of the respiratory, intestinal and genitourinary tracts, tears, saliva, milk, white blood cells, myelomas and the like, biological fluids such as cell culture supernatants, fixed tissue specimens and fixed cell specimens Any substance which can be prepared and tested with the assay formats described in the present invention are contemplated to be within the scope of the present invention.

The methods of the present invention are useful to carry out bio-sensing, molecular biological and molecular diagnostic analyses including proteomics, genomics, drug screening/identification, genotyping, gene expression, DNA diagnostics (cancer, genetic diseases, infectious diseases), infectious agent detection, bioterror agent detection, and for human identification and forensic applications. By way of example, the present invention is useful for genotyping single point mutations in the same manner as known assays, such as a plasma based assay, Taqman, restriction digestion of PCR products, calorimetric mini-sequencing assay, radioactive labeled based solid-phase mini sequencing technique, allele-specific oligonucleotide (ASO), and single strand conformation polymorphism (SSCP).

All references cited in this disclosure are incorporated by reference to the same extent as if each reference had been incorporated by reference in its entirety individually.

While the invention has been described in detail and with reference to specific embodiments thereof, it will be apparent to one skilled in the art that various variations and modifications can be made therein without departing from the spirit and scope thereof. All such variations and modifications are intended to be included within the scope of this disclosure and the present invention and protected by the following claims.

What is claimed is:

1. A method of detecting an analyte in a sample, comprising exposing a sample to a sequence specific polymer under conditions such that an analyte in the sample binds to the sequence specific polymer, wherein binding of the analyte to the sequence specific polymer results in a change in a property of the sequence specific polymer that is transduced to a response transduction medium which generates a detectable response, and detection of said response.

2. The method of claim 1, wherein the sequence specific polymer is a non-biopolymer.

3. The method of claim 1, wherein the sequence specific polymer is a biopolymer.

4. The method of claim 1, wherein the sequence specific polymer is a poly(N-substituted glycine).

5. The method of claim 1, wherein the property of the sequence specific polymer which is changed is a chemical, thermodynamic, mechanical, thermal, electromagnetic or quantum mechanical property.

6. The method of claim 1, wherein the detectable response is a signal which is an electrical, mechanical, optical, acoustic or thermal signal.

7. The method of claim 1, wherein the detectable response is actuation of a process.

8. The method of claim 7, wherein the process is change in delivery rate of a drug or actuation of a valve.

9. The method of claim 1, wherein the sample is a gas, a liquid, a supercritical fluid, or a semisolid.

10. The method of claim 1, wherein the response transduction medium is a cantilever or deflectable membrane.

11. The method of claim 1, wherein the sample is exposed to an array of a plurality of sequence specific polymers, each sequence specific polymer associated with a separate response transduction medium.

12. A method of detecting an analyte in a sample, comprising exposing a sample to a sequence specific polymer under conditions such that an analyte in the sample binds to the sequence specific polymer, wherein binding of the analyte to the sequence specific polymer results in a high local concentration of analyte which causes a change in a property of one or both of:
(a) an ingredient other than the sequence specific polymer in a sequence specific polymer-containing composition, or
(b) a component of a response transduction medium;
such that the change in property is transduced to the response transduction medium which generates a detectable response, and
detection of said response.

13. The method of claim 12, wherein the sequence specific polymer is a non-biopolymer.

14. The method of claim 12, wherein the sequence specific polymer is a biopolymer.

15. A detection device comprising a sequence specific polymer and a response transduction medium, the device having the characteristic that binding of an analyte to the sequence specific polymer causes a change in a property of the sequence specific polymer that is transduced to the response transduction medium and results in a detectable signal.

16. The detection device of claim 15, wherein the sequence specific polymer is a non-biopolymer.

17. The detection device of claim 15, wherein the sequence specific polymer is a biopolymer.

18. The detection device of claim 15, wherein the sequence specific polymer is present at a liquid-gas or liquid-liquid interface.

19. The detection device of claim 15, wherein the sequence specific polymer is on a solid or semisolid surface.

20. The detection device of claim 15, wherein the sequence specific polymer is on a deflectable element.

21. The detection device of claim 20, wherein the deflectable element is a membrane or cantilever.

22. The detection device of claim 15, wherein the sequence specific polymer is in or upon an electrically conductive element or a fluorescent medium.

23. The detection device of claim 15, wherein the sequence specific polymer is in association with a medium such that binding of the analyte to the sequence specific polymer induces nucleation of condensed matter.

24. The detection device of claim 15, wherein the sequence specific polymer has side chains R pendant from a structurally repetitive backbone.

25. The detection device of claim 24, wherein one or more of the pendant side chains R have different structures.

26. The detection device of claim 24, wherein the sequence specific polymer comprises a structure selected from the group consisting of —NR—CH₂—CO—, —O—SiCH₃R—, —NH—CH₂—CHR—CO—, —CH₂—CHR—O— and —NR—CH₂—C₆H₄—CO—.

27. The detection device of claim 24, wherein the sequence specific polymer comprises a hydrophobic backbone.

28. The detection device of claim 27, wherein the sequence specific polymer comprises a structure selected from the group consisting of —CH₂—CHR—, —CH₂—CH₂—CHR—, —CF₂—CFR— and —CF₂—CF₂—CFR—.

29. The detection device of claim 24, wherein the sequence specific polymer comprises a hydrophilic backbone.

30. The detection device of claim 29, wherein the sequence specific polymer comprises the structure —CH₂—CH(CO—NHR)—.

31. The detection device of claim 24, wherein the sequence specific polymer has the structure —NR—CH₂—CO—.

32. The detection device of claim 15, which comprises an array of a plurality of sequence specific polymers, each sequence specific polymer associated with a separate response transduction medium.

33. A detection device comprising a sequence specific polymer and a response transduction medium, the device having the characteristic that binding of an analyte to the sequence specific polymer results in a high local concentration of analyte which causes a change in a property of one or both of:
(a) an ingredient other than the sequence specific polymer in a sequence specific polymer-containing composition, or
(b) a component of the response transduction medium;
such that the change in property is transduced to the response transduction medium and results in a detectable signal.

34. The detection device of claim 33, wherein the sequence specific polymer is a non-biopolymer.

35. The detection device of claim 33, wherein the sequence specific polymer is a biopolymer.

36. A sequence specific polymer for use in analyte detection, wherein binding of an analyte to the sequence specific polymer causes a change in the sequence specific polymer which is capable of being transduced to a detectable response.

37. The sequence specific polymer of claim 36, wherein the sequence specific polymer is a non-biopolymer.

38. The sequence specific polymer of claim 36, wherein the sequence specific polymer is a biopolymer.

39. The sequence specific polymer of claim 36, wherein the polymer comprises a structure selected from the group consisting of —NR—CH₂—CO—, —O—SiCH₃R—, —NH—CH₂—CHR—CO—, —CH₂—CHR—O—, —NR—CH₂—C₆H₄—CO—, —CH₂—CHR—, —CH₂—CH₂—CHR—, —CF₂—CFR—, —CF₂—CF₂—CFR— and —CH₂—CH(CO—NHR)—, wherein R is a pendant side chain.

40. The sequence specific polymer of claim 36, wherein the polymer comprises the structure —NR—CH₂—CO—, wherein R is a pendant side chain.

41. A method for identifying a sequence specific polymer, the method comprising:
(a) providing a preselected target analyte molecule;
(b) providing a plurality of monomers;
(c) identifying monomers that individually, or in combination, interact with the target analyte or portion of the target analyte;
(d) assembling the monomers identified in c) into at least one sequence specific polymer comprising a specific sequence and length of monomers, wherein the polymer comprises at least two monomers;
(e) contacting the sequence specific polymer with the preselected target analyte molecule; and
(f) identifying a sequence specific polymer that interacts with the preselected target analyte molecule.

42. The method of claim 41, wherein the sequence specific polymer is a non-biopolymer.

43. The method of claim 41, wherein the sequence specific polymer is a biopolymer.
